(19) United States
(12) Patent Application Publication
(10) Pub. No.: US 2003/0191419 A1
(43) Pub. Date: Oct. 9, 2003

(54) Protective cover for a cast

(76) Inventors: Mark Melin, Plymouth, MN (US); Cindy Melin, Plymouth, MN (US)

Correspondence Address: CROMPTON, SEAGER & TUFTE, LLC, 1221 NICOLLET AVENUE, SUITE 800, MINNEAPOLIS, MN, 55403-2420, US

(21) Appl. No.: 10/116,262

(22) Filed: Apr. 4, 2002

(51) Int. Cl. A61F 5/00

(52) U.S. Cl. 602/3; 602/62

(57) ABSTRACT

A distal extremity cast cover comprising a sleeve or gaiter portion which may be non-abrasive and which extends distally substantially to the edge of the cast, and a distal extremity covering which overlaps the sleeve or gaiter to a point proximal the wrist or ankle and which may comprise an insulating or water-repellent material. The parts of the cover may be secured by elastic and frictional properties of the materials and/or by elastic bands or pull cords proximate the openings.

### FIELD OF THE INVENTION

The present invention relates to cast covers adapted to cover the exposed hand or foot of a patient and protect the environment from the rough surface of the cast.

### SUMMARY OF THE INVENTION

The present invention pertains to covers for distal extremity casts. An object of the invention is to provide a cover for a cast which is non-damaging to surfaces in which it comes in contact, such as skin, clothing, furniture, walls, or trim. Another object of the invention is to provide a cast covering which provides the patient with protection from temperature extremes on the exposed limb distal of the cast. A further object of the invention is to provide the patient with a water-resistant cast covering to provide the exposed skin distal of the cast with an enhanced opportunity to stay dry. The invention is not limited to these objects and further objects of the invention are disclosed in the specification below.

In a first non-limiting embodiment, the invention is adapted for use with a lower extremity cast. This embodiment of the invention includes a gaiter, which substantially covers the cast and which may extend, for example, from the knee to the metatarsals. This gaiter may be flexible, elastic and/or non-abrasive and may include elastic bands or pull cords at its proximal and distal ends. This embodiment also includes a distal foot covering, which may cover the foot and extend proximal of the ankle. The distal foot covering may be flexible, elastic, and/or non-abrasive and may include an elastic band or pull cord at the proximal end. The distal foot covering may also include water-resistant or insulating material, particularly in a region distal of the metatarsals, such as a polyester fleece material. The gaiter may first be applied over the cast and then the distal foot covering is then applied thereover. The friction between an ankle portion of the gaiter and an ankle portion of the distal foot covering helps retain the cover in place.

In a second non-limiting embodiment, the invention is adapted for use with an upper extremity cast. This embodiment of the invention includes a sleeve, which substantially covers the cast, and which may extend, for example, from the elbow to the metacarpals. The sleeve may have substantially the same qualities as the gaiter as described above. The sleeve may include elastic bands or pull cords at its proximal and distal ends and may include a plurality of holes at the distal end, such as, for example, a thumb hole and a finger hole. The embodiment also includes a distal hand and wrist cover, which may extend proximal of the wrist. The distal hand and wrist cover may have substantially the same qualities and include substantially the same material as the distal foot cover as described above. In a particular embodiment, the insulating or water-resistant material may extend distal of the metacarpals. The sleeve may first be applied over the cast and the distal hand and wrist cover may then be applied thereover. The fiction between the wrist portion of the sleeve and the wrist cover help retain the cover in place.

### BRIEF DESCRIPTION OF THE DRAWINGS

FIG. 1 is a diagrammatic view of a cover for a lower extremity cast showing the cover applied over a cast;

FIG. 2 is a diagrammatic view of the flexible elongate gaiter of the cover of FIG. 1;

FIG. 3 is a diagrammatic view of the distal foot covering of the cover of FIG. 1;

FIG. 4 is a diagrammatic view of a cover for an upper extremity cast showing the cover applied over a cast;

FIG. 5 is a diagrammatic view of the flexible elongate sleeve of the cover of FIG. 4;

FIG. 6 is a diagrammatic view of the distal hand and wrist covering in the form of a glove of the cover of FIG. 4; and

FIG. 7 is a diagrammatic view of the distal hand and wrist covering in the form of a mitten of the cover of FIG. 4.

### DETAILED DESCRIPTION OF THE INVENTION

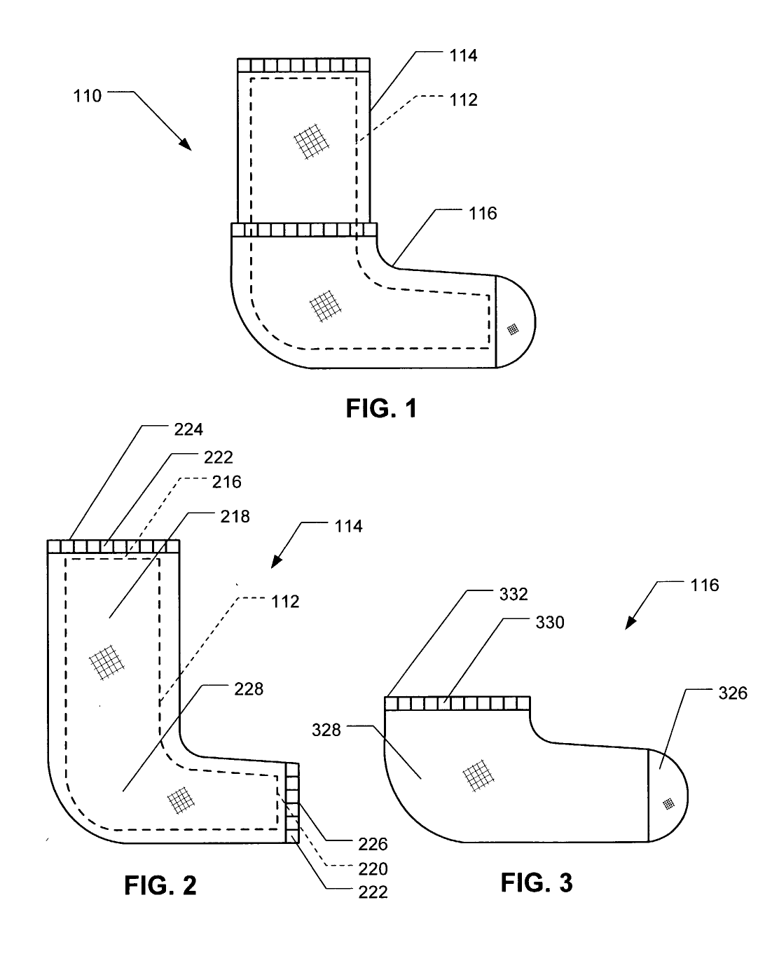

Referring now to the drawings wherein like reference numerals refer to like elements throughout the several drawings, FIG. 1 is a diagrammatic view of a cover 110 for a lower distal extremity cast 112 showing the cover 110 applied over cast 112. The cover includes a gaiter 114 and a distal foot covering 116.

As may be best seen in FIG. 2, gaiter 114 extends over substantially the whole length of cast 112. Gaiter 114 may extend proximally and distally beyond the ends of cast 112 as depicted in FIG. 2 or gaiter 114 may extend a lesser length. At the proximal end 216 of cast 112, the gaiter 114 may extend up to and not beyond the cast 112. It is generally preferred that the gaiter cover the outer surface 218 of cast 112, so the gaiter, or a gaiter and a legging (not shown), will generally extend at least up to proximal end 216. In contrast, because distal extremity covering 116 overlaps gaiter 114, it is not necessary that gaiter 114 extend up to the distal end 220 of the cast 112. However, in the embodiment shown in FIG. 2, gaiter 114 extends distally beyond distal end 220 of cast 112 and in a contemplated embodiment gaiter 114 extends up to distal end 220 of cast 112. Gaiter 114 includes an ankle portion 228.

It may be readily seen that for ease of installation over rigid cast 112, gaiter 114 will be flexible. Gaiter 114 may also be elastic. Having a degree of elasticity in gaiter 114 allows gaiter 114 to accommodate casts 112 of various sizes while providing gaiter 114 an additional means to retain to cast 112.

According to an embodiment of the present invention, gaiter 114 may be non-abrasive. The term non-abrasive is herein understood to mean having a degree of softness or smoothness necessary to not irritate or abrade skin which may come in contact with the gaiter and necessary to allow clothing materials to pass by without catching and necessary to avoid scratching surfaces of furniture, walls or trim. At a minimum, the term non-abrasive means less abrasive than the cast 112. It will be appreciated that a wide variety of materials are non-abrasive as herein defined, of which woven cotton, polyester, and nylon are only non-limiting examples.

The material of which gaiter 114 is made may also have a thickness or pile substantially greater than or equal to the surface roughness of cast 114. The term surface roughness of the cast is herein understood to mean the depth and height to which the actual surface of the cast protrudes below and above a theoretical, perfectly smooth cast surface having the same overall topology as the actual cast surface. The advantages of having this thickness or pile to gaiter 114 may be readily appreciated. Some of these advantages are additional cushioning to protect adjacent surfaces and an enhanced uniformity of appearance. Of course, other thicknesses of material are contemplated, both thicker and thinner than that of the above disclosed embodiment, which, depending on the particular material selected for use in gaiter 114, may provide the above disclosed advantages. For example, in a particular embodiment the material of gaiter 114 is heavy knit cotton, of weight similar to that of a hockey sock.

While, as disclosed above, gaiter 114 may be elastic, this quality is by no means required. For instance, gaiter 114 may also be deformable without being elastic. In these embodiments, but certainly not limited to these embodiments, it may be advantageous to include in gaiter 114 elastic bands or pull cords 222 at the proximal end 224 and the distal end 226 of gaiter 114. The advantages of such bands 222 are readily appreciated. For instance, bands 222 may enhance the retaining power of gaiter 114 to cast 112 or may allow proximal 224 and distal 226 ends of gaiter 114 to cover the exposed cast surface at the proximal 216 and distal 220 ends of cast 112.

Other variations of gaiter 114 are also contemplated. For instance, it may be advantageous to line gaiter 114 with a layer of relatively slick material (not shown), such as a woven nylon or rayon shell, to aid in its application over rough surface 218 of cast 112. An embodiment is also contemplated wherein gaiter 114 includes a plurality of coterminous or overlapping segments, which may advantageously enhance ease of installation.

As may be best seen in FIG. 3, distal foot covering 116 includes a distal extremity portion 326 and an ankle portion 328. Distal foot covering 116 generally extends proximal of ankle portion 228 of gaiter 114, the purpose and advantages of which will be discussed below.

Like gaiter 114, distal foot covering 116 may be elastic or non-abrasive, said qualities having many of the purposes and advantages discussed above with reference to gaiter 114. Distal foot covering 116 may also be insulating. The term insulating is herein understood to mean providing thermal retention including, for example, heat retention capabilities. As used herein, the term insulating may refer to materials having a lower coefficient of thermal conductivity than the material of the gaiter 114, or than conventional weight cotton sock material. Examples of insulating materials include polyester fleece, wool, or a quilted material with a cotton or nylon shell. In a particular embodiment, only the distal extremity portion 326 of distal foot covering 116 includes insulating material. Advantages of including insulating material in the distal foot covering 116 include keeping the foot at a comfortable temperature and protecting the foot from extreme temperatures.

Distal foot covering 116, particularly distal extremity portion 326, may include water-repellent material. The term water-repellent is understood to mean material which is hydrophobic or which does not wick water towards the foot. Examples of water-repellent materials include polyester fleece, Gore-Tex™, nylon, and waxed cotton. Advantages of including water-repellent material in distal foot covering 116 include keeping moisture from the foot and providing a distal foot covering 116 which dries rapidly, particularly in the areas in which it is exposed to the skin of the foot (not shown).

Distal foot covering 116 may include an elastic band or pull cord 330 at the proximate end 332 of the foot cover. Elastic band or pull cord 330 would enhance the retention capabilities of distal foot covering 116.

It is also contemplated that distal foot covering 116 and gaiter 114 would be washable. This would enhance the ability of the patient to maintain cover 110 in a hygienic state.

Gaiter 114 and distal foot covering 116 may be made by conventional manufacturing techniques well known to those of skill in the art.

In use, foot cover 110 may be applied in the following manner, as disclosed in this particular embodiment. Gaiter 114 may first be applied on cast 112 such that proximal end 224 of gaiter 114 is at or near proximal end 216 of cast 112. It may be desirable to have proximal end 224 of gaiter 114 extend up to or beyond proximal end 216 of cast 112. Distal foot covering 116 may then be applied such that ankle portion 328 of distal foot covering 116 overlaps ankle portion 228 of gaiter 114. The friction between the two ankle portions 116 and 228 may help retain distal foot covering 116 in place. Other uses, such as first applying distal foot covering 116 and then applying gaiter 114, are contemplated.

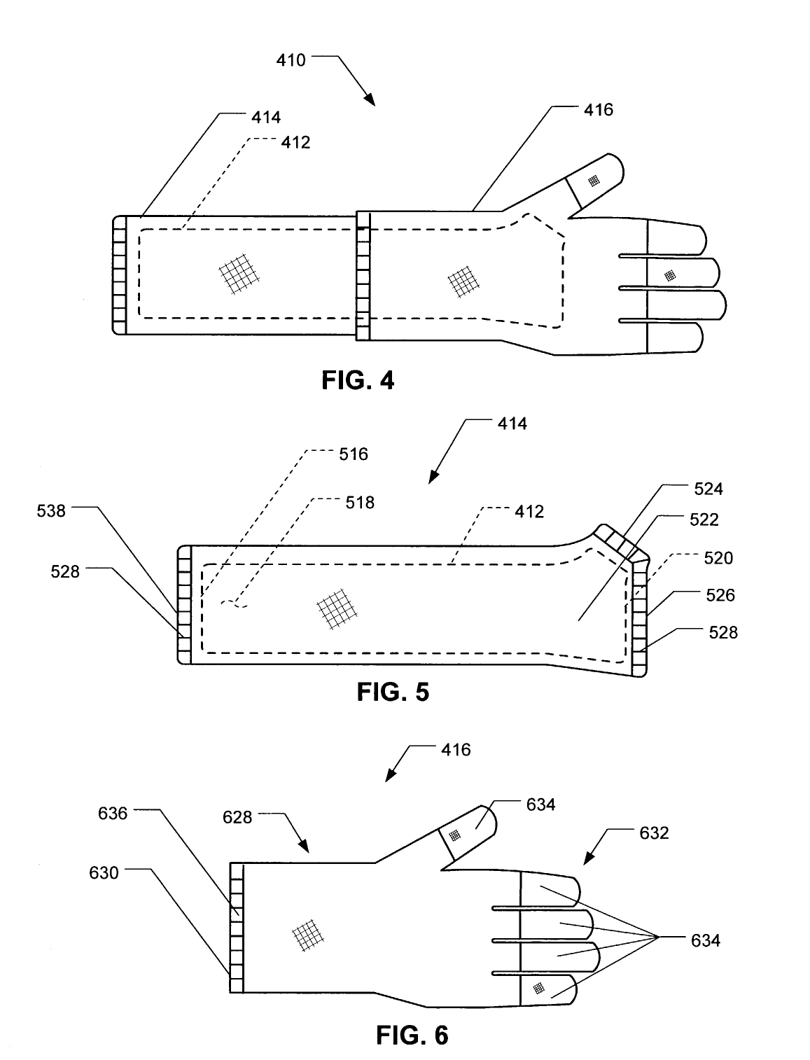

Refer now to FIG. 4 which is a diagrammatic view of a cover 410 for an upper distal extremity cast 412 with the cover 410 applied over cast 412. The cover includes a sleeve 414 and a distal hand and wrist covering 416.

As may be best seen in FIG. 5, sleeve 414 extends over substantially the whole length of cast 412. Sleeve 414 may extend proximally and distally beyond the ends of cast 412 as depicted in FIG. 5 or may extend a lesser length. As an example, at the proximal end 516 of cast 412, sleeve 414 may extend up to and not beyond cast 412. It is generally preferred that sleeve 414 cover the outer surface 518 of cast 412. Therefore, sleeve 414 or a plurality of sleeves (not shown), will generally extend at least up to proximal end 516 of cast 412. In contrast, because distal hand and wrist covering 416 overlaps sleeve 414 at the distal end of the cast, it is not necessary that sleeve 414 extend up to distal end 520 of cast 412. However, in the embodiment shown in FIG. 5, sleeve 414 extends distally beyond distal end 520 of cast 412, and in a contemplated embodiment sleeve 414 extends up to distal end 520 of cast 412. In the embodiment of FIG. 5, sleeve 414 includes a wrist portion 522, a thumb opening 524 and a finger opening 526.

It will be appreciated that sleeve 414 may include a different number of distal openings than that of the previous embodiment. For instance, if sleeve 414 does not extend to distal end 520 of cast 412, sleeve 414 may include a single distal opening (not shown). An embodiment is also contemplated in which sleeve 414 may include four separate finger openings (not shown). A further embodiment is contemplated where sleeve 414 includes a plurality of coterminous or overlapping segments, which may advantageously ease application of sleeve 414.

While, as disclosed above, sleeve 414 may be elastic, this quality is by no means required. For instance, sleeve 414 may also be deformable without being elastic. In these embodiments, but certainly not limited to these embodiments, it may be advantageous to include in sleeve 414 elastic bands or pull cords 528 at the proximal end 538 and the distal end 526 of sleeve 414. The advantages of such bands 528 are readily appreciated. For instance, bands 528 may enhance the retaining power of sleeve 414 to cast 412 or may allow proximal 538 and distal 526 ends of sleeve 414 to cover the exposed cast surface at the proximal 516 and distal 520 ends of cast 412.

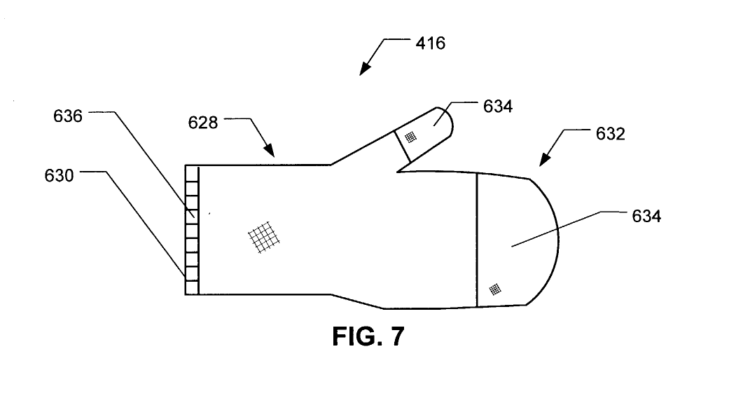

FIGS. 6 and 7 show distal hand and wrist covering 416. Covering 416 includes a wrist portion 628, a proximal wrist end 630, and a distal digit portion 632. Distal digit portion 632 may include separate and separately articulated digit lumens 634 such as a glove as shown in FIG. 6, or shared lumens 634 such as a mitten as shown in FIG. 7. Distal hand and wrist covering 416 may include elastic band or pull cord 636 to enhance the retaining power of covering 416.

Sleeve 414 and distal hand and wrist covering 416 may include the materials discussed above with reference to gaiter 114 and distal foot covering 116, respectively. Because the materials, and the purposes and advantages for them are similar to the materials and purposes of gaiter 114 and distal foot covering 116, further discussion is unnecessary.

In use, cover 410 may be applied in the following manner, as contemplated in this particular embodiment. Sleeve 414 may be first applied on cast 412 such that proximal end 538 of sleeve 414 is at or near proximal end 516 of cast 412. It may be desirable to have proximal end 536 of sleeve 414 extend up to or beyond proximal end 516 of cast 412. Distal hand and wrist covering 416 may then be applied such that wrist portion 628 of covering 416 overlaps wrist portion 522 of sleeve 414. The friction between the two wrist portions 522 and 628 will help retain distal hand and wrist portion 416 in place. Other uses, such as first applying distal hand and wrist covering 416 and then applying sleeve 414, are also contemplated.

Numerous characteristics and advantages of the invention covered by this document have been set forth in the foregoing description. It will be understood, however, that this disclosure is in many respects, only illustrative. Changes may be made in details without exceeding the scope of the invention. The scope of the invention is of course not limited to the foregoing illustrative embodiments but is rather defined by the appended claims.

What is claimed is:

1. A cover for use with a distal limb cast, comprising:
a flexible elongate sleeve having a proximal end and a distal end and a lumen extending from the proximal end to the distal end, wherein the elongate sleeve is sized to extend over substantially the whole length of the distal limb cast; and
a distal extremity covering having a distal extremity end and an open proximal end, wherein the proximal end of the covering extends proximally over the distal end of the elongate sleeve.

2. The cover of claim 1 wherein the flexible elongate sleeve is non-abrasive.

3. The cover of claim 1 wherein the flexible elongate sleeve comprises fabric.

4. The cover of claim 1 wherein the lumen defines a wall having a thickness, wherein the wall thickness is at least as great as the surface roughness of the cast.

5. The cover of claim 1 wherein the flexible elongate sleeve comprises heavy knit cotton.

6. The cover of claim 1 wherein the flexible elongate sleeve further comprises elastic bands or pull cords at the proximal and distal ends.

7. The cover of claim 1 wherein the distal extremity end of the distal extremity covering is closed.

8. The cover of claim 1 wherein the distal extremity covering comprises an insulating material.

9. The cover of claim 1 wherein the distal extremity covering comprises a water-repellent material.

10. The cover of claim 1 wherein the distal extremity covering comprises an elastic material.

11. The cover of claim 1 wherein the distal extremity covering comprises a polyester fleece material.

12. The cover of claim 1 wherein the distal extremity covering further comprises an elastic band or pull cord at the proximal end.

13. The cover of claim 1 wherein the elongate sleeve and the distal extremity covering are washable.

14. A cover for use with a lower extremity cast, comprising:
a flexible elongate gaiter having a proximal knee end, an ankle portion, a distal metatarsal end, and a lumen extending between the knee end and the metatarsal end, wherein the lumen is sized to receive the lower extremity cast; and
a distal foot covering having a distal extremity portion, an ankle portion, a proximal ankle end and a lumen opening at the proximal ankle end, wherein the ankle end is sized to receive the distal metatarsal end of the flexible elongate gaiter and the ankle portion of the distal foot covering overlaps the ankle portion of the flexible elongate gaiter.

15. The cover of claim 14, wherein the flexible elongate gaiter further comprises elastic bands or pull cords at the proximal knee end and at the distal metatarsal end, and wherein the distal foot covering further comprises an elastic band or pull cord at the proximal ankle end.

16. The cover of claim 14, wherein the distal extremity portion of the distal foot covering comprises an insulated, water-resistant material, and wherein the flexible elongate gaiter comprises a non-abrasive material.

17. A cover for use with an upper extremity cast, comprising:
a flexible elongate gaiter having a proximal elbow end, a wrist portion, a distal base-of-digits end, a plurality of openings at the distal metacarpal end, an arm opening at the proximal elbow end, and a lumen extending between the openings; and
a distal hand and wrist covering having a distal digit portion, a wrist portion, a proximal wrist end, and a lumen opening at the proximal wrist end, wherein the proximal wrist end is sized to fit over the distal end of the flexible elongate gaiter and the wrist portion of the distal hand and wrist cover overlaps the wrist portion of the flexible elongate gaiter.

18. The cover of claim 17, wherein the distal hand and wrist covering comprises separate digit lumens at the distal digit portion, and wherein the plurality of openings at the distal metacarpal end of the gaiter comprise a thumb opening and a finger opening.

19. The cover of claim 17, wherein the flexible elongate gaiter further comprises elastic bands or pull cords at the proximal elbow end and at the distal metacarpal end, and wherein the distal hand and wrist cover further comprises an elastic band or pull cord at the proximal wrist end.

20. The cover of claim 17, wherein the distal digit portion of the distal hand and wrist covering comprises an insulated, water-resistant material, and wherein the flexible elongate sleeve comprises a non-abrasive material.
